(19) United States
(12) Patent Application Publication
(10) Pub. No.: US 2013/0084267 A1
(43) Pub. Date: Apr. 4, 2013

(54) METHOD FOR STEM CELL DIFFERENTIATION

(71) Applicants: Nicholas Maxwell Fisk, Queensland (AU); Ernst Jurgen Wolvetang, St. Lucia (AU); Rebecca Anne Pelekanos, Herston (AU)

(72) Inventors: Nicholas Maxwell Fisk, Queensland (AU); Ernst Jurgen Wolvetang, St. Lucia (AU); Rebecca Anne Pelekanos, Herston (AU)

(73) Assignee: THE UNIVERSITY OF QUEENSLAND, Queensland (AU)

(21) Appl. No.: 13/575,493

(22) PCT Filed: Jan. 28, 2011
(86) PCT No.: PCT/AU2011/000087

§ 371 Date: Oct. 11, 2012

(30) Foreign Application Priority Data
Jan. 28, 2010 (AU) 2010900330

(51) Int. Cl. C12N 5/071; A61K 35/12

(52) U.S. Cl. CPC C12N 5/0662; A61K 35/12
USPC 424/93.7; 435/377

(57) ABSTRACT

The present invention relates generally to a method for generating mesenchymal stem cells from pluripotent cells, the method comprising (i) differentiating a population of pluripotent stem cells in the presence of an inhibitor of endogenous activin and TGF-β signalling and (ii) passaging the cells differentiated in step (i) in the presence of a mesenchymal stem cell medium for a time and under conditions sufficient to produce mesenchymal stem cells. The present invention also relates to mesenchymal stem cells produced by the methods of the present invention and uses thereof.

### FIELD OF THE INVENTION

The present invention relates to methods for generating mesenchymal stem cells from pluripotent cells.

### BACKGROUND OF THE INVENTION

Mesenchymal stem/stromal cells (MSC) are limited by their rarity in adult organs, their heterogeneity, and the need to harvest by invasive procedures. Accordingly, attention has focused on deriving MSC from human embryonic stem cells (hESC) as a potentially robust, scalable system for generating homogenous cells suitable for cell therapy. A variety of techniques have been used to direct hESC into MSC-like cells, ranging from untranslatable approaches involving immortalisation or mouse feeders, through to cumbersome physical or epitope selection.

Boyd et al. (2009) Tissue Engineering: Part A 15(8):1897-1907 developed a method to differentiate hESC into mesodermal progenitors using a 30-day culture in epithelial culture media, followed by 2-3 passages to induce epithelial to mesenchymal transition (EMT). This resulted in cells with decreased pluripotency marker expression and increased mesodermal/MSC marker expression. Furthermore, the ES-MSC were able to differentiate along mesodermal lineages and remodel a collagen lattice, similar to MSC.

The TGF-β pathway inhibitor, SB431542, has been used to differentiate hESC into several cell types including epithelium (Watabe et al. (2003) The Journal of Cell Biology 163(6):1303-1311). Recently, two groups demonstrated that bFGF/TGF-β pathways are required to keep hESC in a pluripotent state (Vanier et al. (2005). J. Cell Sci. 118:4495-4509; Vallier et al. (2009) Development 136:1339-1349; Xu et al. (2008) Cell Stem Cell 3:196-206). The inhibition of the TGF-β pathway using a synthetic inhibitor, SB431542, led to hESC differentiation by inhibiting SMAD2/3 phosphorylation and subsequent decrease in NANOG promoter activity (Xu et al. 2008).

### SUMMARY OF THE INVENTION

In a first aspect of the present invention there is provided a method for generating mesenchymal stem cells from a population of embryonic stem cells (ESC) or induced pluripotent stem cells (iPS), the method comprising:

(i) differentiating a population of ESC or iPS attached to a surface of a culture vessel by exposing the cells to an inhibitor of endogenous activin and TGF-β signalling to produce a monolayer of cells comprising epithelial cell-like morphology attached to the surface of the culture vessel; and

(ii) passaging the cells differentiated in step (i) in the presence of a mesenchymal stem cell medium for a time and under conditions sufficient to produce mesenchymal stem cells.

In a second aspect of the present invention there is provided a method for generating mesenchymal stem cells from a population of ESC or iPS, the method comprising:

(i) differentiating a population of ESC or iPS in the presence of an inhibitor of endogenous activin and TGF-β signalling under conditions sufficient to inhibit formation of embryoid bodies (EB); and

(ii) passaging the cells differentiated in step (i) in the presence of a mesenchymal stem cell medium for a time and under conditions sufficient to produce mesenchymal stem cells. In some embodiments, the population of ESC or IFS is attached to a surface of a culture vessel.

In some embodiments, the inhibitor of endogenous activin and TGF-β signalling is 4-[4-(1,3-benzodioxol-5-yl)-5-(2-pyridinyl)-1H-imidazol-2-yl]-benzamide (SB431542). In some embodiments, the ESC or iPS is differentiated in step (i) in the presence of an attachment factor.

In some embodiments, step (i) comprises differentiating a population of iPS.

In a third aspect of the present invention there is provided a mesenchymal cell or population of mesenchymal cells generated by the method of the present invention, as herein described.

In a fourth aspect of the present invention there is provided a pharmaceutical composition comprising a mesenchymal cell or population of mesenchymal cells generated by the method according to the present invention, as herein described.

In a fifth aspect of the present invention there is provided a tissue matrix comprising a mesenchymal cell or population of mesenchymal cells generated by the method according to the present invention, as herein described.

In a sixth aspect of the present invention there is provided a mesenchymal cell or population of mesenchymal cells generated by the method according to the first and second aspects of the present invention, the pharmaceutical composition according to the fourth aspect of the present invention, or the tissue matrix according to the fifth aspect of the present invention, for use in human therapy.

In a seventh aspect of the present invention there is provided a mesenchymal cell or population of mesenchymal cells generated by the method according to the first and second aspects of the present invention, the pharmaceutical composition according to the fourth aspect of the present invention, or the tissue matrix according to the fifth aspect of the present invention, for veterinary use.

### BRIEF DESCRIPTION OF THE DRAWINGS

FIG. 1A shows the morphology of hECS and iPSC cultured in the presence of SB431542. (A) hESC remain as tightly packed colonies of small cells when cultured in normal growth conditions. After 10 days incubation with the SB431542 inhibitor, the cells have differentiated into an epithelial-like monolayer. (B) When these cells are transferred to MSC media, they again change morphology and become fibroblastic. (C) MSC also have a fibroblast-like morphology. (D) hiPSC line ES4CL1 also differentiates though an epithelial-like morphology into MSC-like cells with this SB431542 inhibitor culture method.

FIG. 1B shows morphology of iPSC and ESC undergoing differentiation through the inhibitor method. Morphological depiction of iPSC (A, C and E) and ESC (B, D and F) undergoing differentiation through the inhibitor method (i.e., when differentiated in the presence of SB431542) from undifferentiated cells (A and B), after 10 days in SB431542 (C and D) and after 5-6 passages in fMSC medium (E and F). Abbreviations: iPSC (induced pluripotent stem cells), ESC (embryonic stem cells), fMSC (fetal mesenchymal stem cell).

FIG. 1C shows immunofluorescence investigation of epithelial to mesenchymal transition (EMT) during derivation of iPS-MSC (inhibitor method). Immunofluorescence revealed that undifferentiated iPSC expressed E-cadherin (red) throughout the colony whereas N-cadherin (green) expression was limited to the periphery of the cell colony (top row) where spontaneously differentiating cells are localised. iPS-MSC (inhibitor) at MP2 showed no expression of E-cadherin but expressed N-cadherin outside the nucleus of all cells viewed, as seen in definitive EMT (bottom row). Thus, with differentiation, the classic E-cadherin to N-cadherin switch was observed, as in definitive EMT. Abbreviations: iPSC (induced pluripotent stem cells), iPS-MSC (induced pluripotent stem cell-derived MSC), MSC (mesenchymal stem/stromal cells), MP (mesenchymal passage), inhibitor (SB431542; inhibitor method).

FIG. 2 shows the immunophenotype of ES-MSC. ES-MSC display an immunophenotype similar to fetal MSC having the phenotypic markers CD73⁺, CD105⁺, CD90⁺, HLA-ABC low⁺, HLA-DR⁻ and CD31⁻. In contrast, the original hESC line, MEL1, had phenotypic markers CD105⁺, CD90⁺, HLA-ABC low⁻, CD73⁻, HLA-DR⁻ and CD31⁻.

FIG. 3 shows in vitro osteogenic differentiation of ES-MSC. ES-MSC demonstrate comparable osteogenic differentiation to fetal bone marrow MSC (fMSC) as determined by (A) Alizarin red or (B) von Kossa staining. After 21 days in osteogenic induction media (+, upper panels) or normal growth media (−, lower panels) cells were stained to determine mineralization and calcium accumulation.

FIG. 4 shows immunofluorescence marker analysis of ES-MSC cultures. ES-MSC, fetal MSC (fMSC) and the hESC line MEL1 were stained for expression of mesodermal markers Collagen I and Vimentin, the hematopoietic marker CD45 and the pluripotent stem cell marker Oct4. ES-MSC and fMSC were positive for Collagen I and Vimentin, and were negative for CD45 and Oct4. hESC were negative for the lineage specific markers, and positive for the Oct4.

FIG. 5 shows Human Nuclear Antigen expression by ES-MSC. ES-MSC were stained with the Human Nuclear Antigen Antibody to ensure fibroblast-like cells were human in origin and not Contaminating mouse embryonic fibroblasts (MEF, the feeder layer used in culturing undifferentiated hESC and iPSC).

FIG. 6 shows the immunophenotype of iPC-MSC. The human iPSC line ES4CL1 differentiated into MSC using SB431542 displayed an immunophenotype similar to MSC: CD29⁺, CD13⁺, CD44⁺, CD146⁺, CD73⁺, CD105⁺, CD90⁺, HLA-ABC low⁺, HLA-DR⁻, CD14⁻, CD45⁻, CD11b⁻, CD24⁻, CD31⁻, CD34⁻, CD11T.

FIGS. 7A-7C shows the cell surface immunophenotype of fetal MSC (fMSC), iPS-MSC (SB431542), iPS-MSC (embryoid body method) and iPSC: clinically-defined MSC marker and other common positive and negative marker expression. iPS-MSC expressed positive MSC markers (CD73, CD90 and CD105) at similar levels to fMSC. All MSC samples lacked expression of macrophage and monocyte markers (CD11b and CD14), human leukocyte marker (HLA-DR) and broad hematopoietic markers (CD45) and broad hematopoietic progenitor marker (CD34). iPS-MSC derived by culturing in the presence of SB431542 and fMSC also expressed other markers common to MSC including CD29, CD13, CD44 and CD146. Also consistent with criteria for defining MSC, iPS-MSC expressed low levels of HLA-ABC and completely lacked expression of HLA-DR. fMSC and iPS-MSC derived by culturing in the presence of SB431542 and through formation of embryoid bodies (EB) lacked expression of the ESC and pericyte marker CD24, which was expressed positively by the iPSC sample as expected. The endothelial marker, CD31 and the primitive haematopoietic and progenitor cell marker, CD117, were also not expressed by fMSC, iPS-MSC (SB431542), iPS-MSC (EB) and iPS. fMSC (red histogram; 1^{st} column), iPS-MSC (SB431542; blue histogram; 2^{nd} column), iPS-MSC (EB; green histogram; 3^{rd} column) and undifferentiated iPSC (orange histogram; 4^{th} column) were stained with fluorophore-conjugated antibodies, indicated on the x-axis. Open histograms indicate relevant isotype controls for each epitope.

FIG. 8 shows immunofluorescence marker analysis of iPSC and iPS-MSC. iPSC that were differentiated in the presence of SB431542 were stained for expression of SSEA4, vimentin and pluripotency markers Oct4, Nanog, Stella, SSEA3, Tra 1-60 and Tra 1-81. Differentiation of iPSC in the presence of SB431542 resulted in the decreased expression of Oct4, Nanog, Stella, SSEA3, Tra 1-60 and Tra 1-81. Nuclear expression of SSEA4 was observed by iPSC colonies and iPS-MSC (SB431542) at MP2. Cells gained expression of a mesodermal marker, vimentin during differentiation from iPSC to iPS-MSC in the presence of SB431542, indicating that iPSC had undergone differentiation into iPS-MSC. Nuclei were counter-stained with dapi (blue).

FIG. 9 shows Mesodermal differentiation of fMSC, ES- and iPS-MSC (inhibitor and EB methods)

Mesodermal differentiation of fMSC, iPS-MSC and ES-MSC (inhibitor and EB) after 28 days differentiation medium, stained with von Kossa, Alizarin Red S, Oil Red O and PAS. Microscopy magnificiation: ×100 (von Kossa and Alizarin Red S staining) and ×200 (Oil Red O and PAS staining), scale bars represent 50 and 25 μm respectively. Abbreviations: MS C (mesenchymal stem cell) iPS-MSC (induced pluripotent stem cell-derived MSC), ES-MSC (embryonic stem cell-derived MSC), EB (Embryoid Body), fMSC (fetal MSC), PAS (periodic acid schiff).

FIG. 10 shows karyotypic assessment of fMSC, iPS-MSC (inhibitor and EB method)

Chromosome metaphase spreads showing normal karyotype of (A) fMSC (46XY), (B) iPS-MSC (inhibitor) (46XX) and (C) iPS-MSC (EB) (46XX), analysed at early and late passage (MP11-12 shown). Abbreviations: MSC (mesenchymal stem/stromal cell), fMSC (fetal MSC), iPS-MSC (induced pluripotent stem cell-derived MSC), EB (embryoid body), MP (mesenchymal passage).

FIG. 11 shows assessment of tumourogenicity of iPSC and iPS-MSC through teratoma assay

No teratomas formed in animals injected with iPS-MSC (n=3) nine weeks after injection. In contrast, two teratomas formed in animals injected with iPSC. These grew to 8 mm diameter seven weeks after injection (n=3). Histological analysis of haematoxylin and eosin stained teratoma sections reveals the presence of cells derived from the 3 germ layers (top and bottom row): (B and E) mesoderm (arrows indicate primitive cartilage), (C and F) endoderm (arrows indicate epithelium) and (D and G) ectoderm (arrows indicate neural rosettes). Abbreviations: MSC (mesenchymal stem cell), fMSC (fetal MSC), iPS-MSC (induced pluripotent stem cellderived MSC).

FIG. 12 shows the gene expression analysis of MEL1-derived MSC induced by SB431542 at different stages. The pluripotent hESC line, MEL1, was cultured in KOSR medium with 10 μM SB431542 treatment. After 10 days treatment, MEL1 cells were subcultured in MSC medium for differentiating MSC cells. The MEL1-derived MSC are indicated as MEL1-MSC P2. (“P2” indicates second passages). SB431542 treated cells and MEL1-MSC P2 were analyzed for gene expression. (a-d) OCT4, SOX2, MYST2 and EPCAM genes associated with iPSC pluripotency. (e-h) NCAM, MSX2, LEFTY1 and BMP4 genes expressed by cell lineages from the mesoderm. (i-j) PAX6 gene is expressed by cell lineages from the ectoderm and CDX2 gene is expressed by cell lineages from the trophectoderm. (k) GATA gene expressed by cell lineages from the endoderm. (l-o) Genes expressed by MSC (positive in CD29, CD73 and negative in the CD117, CD133). Gene expression was normalized to GAPDH. All experiments represent duplicates.

FIG. 13 shows the gene expression analysis of MR90-derived MSC induced by SB431542 at different stages. Pluripotent iPSC, MR90, were cultured in KOSR medium with 10 μM SB431542 treatment for 10 days. After 10 days treatment, MR90 cells were subcultured in the MSC medium for differentiating MSC cells. MEL1-derived MSC are indicated as MR90-MSC P0 (passage 0). The SB431542 treated cells and MR90-MSC P0 were analyzed for gene expression. (a-d) OCT4, SOX2, MYST2 and EPCAM genes associated with iPSC pluripotency. (e-h) NCAM, MSX2, LEFTY1 and BMP4 genes expressed by cell lineages from the mesoderm. (i-j) PAX6 gene is expressed by cell lineages from the ectoderm and CDX2 gene is expressed by cell lineages from the trophectoderm. (k) GATA gene expressed by cell lineages from the endoderm. (l-o) Genes expressed by MSC (positive in CD29, CD73 and negative in the CD117, CD133). Gene expression was normalized to GAPDH. All experiments represent duplicates.

FIG. 14 shows the flow cytometry analysis of the EPCAM expression level in the SB431542-treated MR90 at 10 days. (a) the isotype Ab control. (b-c) The 45% EPCAM+ MR90 cell population in the mTESR culture.

FIG. 15 shows the gene expression profile of SB431542-induced ESC/iPSC differentiation. (A, B) The heat map of mRNA expression level was assayed by qRT-PCR array. ESC/iPSC cells were cultured in KOSR condition medium with 10 μM SB431542. After 10 days treatment, cells were subcultured into MSC medium to differentiate cells into MSC. RNA was extracted from MEL1 and MR90 cells at day 10. Data showed the selected genes defining three germ layers and MSC markers. (C) Relative mRNA transcripts folds change of quantitative RT-PCR analysis in the indicated cell subsets. All the gene expression level was normalized to GAPDH mRNA level and compared to genes level in the mTESR culture condition.

### DETAILED DESCRIPTION

In this specification the term “population of pluripotent cells” is intended to mean one or more cells capable of differentiating into a committed cell lineage. Pluripotent cells according to the present invention include, but are not limited to, human embryonic stem cells (hESC) and induced pluripotent stem cells (iPS or iPSC).

Induced pluripotent stem cells, commonly abbreviated as iPS are a type of pluripotent stem cell artificially derived from a non-pluripotent cell, typically an adult somatic cell, by inducing a “forced” expression of certain genes (e.g. Yu et al. (2007) Science 318(5858):1917-20). Examples of iPS include, but are not limited to, ESCL and MR90-series cell lines ESCL1, ESCL2, ESCL3, ESCL4, MR90C2 and MR90C4 (WiCell Research Institute).

Induced Pluripotent Stem Cells are believed to be identical to natural pluripotent stem cells, such as embryonic stem (ES) cells in many respects, such as the expression of certain stem cell genes and proteins, chromatin methylation patterns, doubling time, embryoid body formation, teratoma formation, viable chimera formation, and potency and differentiability.

In this specification the term “SB431542” is intended to mean an inhibitor of the TGF-β1 activin receptor-like kinases (ALKs). It is a selective and potent inhibitor of ALK-4, -5 and -7. SB431542 inhibits endogenous activin and TGF-β signaling without affecting more divergent BMP signaling utilizing ALK-1, -2, -3, and -6 (Inman et al. (2002) Mol. Pharmacol. 62:65-74; Laping et al. (2002) Mol. Pharmacol 62:58-64). an inhibitor of the TGF-β1 activin receptor-like kinases (ALKs). It is a synthetic compound which is a potent and selective inhibitor of ALK-4, -5 and -7. SB431542 is also known as 4-[4-(1,3-benzodioxol-5-yl)-5-(2-pyridinyl)-1H-imidazol-2-yl]-benzamide. SB431542 also has the chemical structure of Formula I, below:

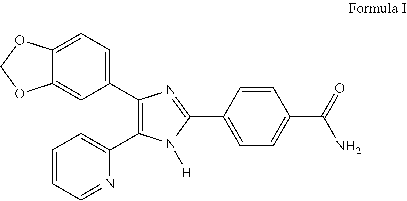 [SMILES: NC(=O)c1ccc(-c2nc(-c3ccc4c(c3)OCO4)c(-c3ccccn3)[nH]2)cc1]

In this specification “mesenchymal stem cells” (or MSCs) are multipotent stem cells that can differentiate into a variety of cell types. Cell types that MSCs have been shown to differentiate into in vitro or in vivo include osteoblasts, chondrocytes and adipocytes. Mesenchymal stem cells are characterized morphologically by a small cell body with a few cell processes that are long and thin. The cell body contains a large, round nucleus with a prominent nucleolus which is surrounded by finely dispersed chromatin particles, giving the nucleus a clear appearance. The remainder of the cell body contains a small amount of Golgi apparatus, rough endoplasmic reticulum, mitochondria, and polyribosomes. The cells, which are long and thin, are widely dispersed and the adjacent extracellular matrix is populated by a few reticular fibrils but is devoid of the other types of collagen fibrils.

Based on the observation that epithelial to mesenchymal transition (EMT) occurs during spontaneous and directed hESC differentiation, the inventor(s) devised a novel method to induce rapid differentiation into a homogenous epithelial-like monolayer, followed by conventional MSC culture for both hESC and induced pluripotent stem cells (iPSC) to produce mesenchymal cells.

Accordingly, in a first aspect of the present invention, there is provided a method for generating mesenchymal stem cells from a population of embryonic stem cells (ESC) or induced pluripotent stem cells (iPS), the method comprising:

(i) differentiating a population of ESC or iPS attached to a surface of a culture vessel by exposing the cells to an inhibitor of endogenous activin and TGF-β signalling to produce a monolayer of cells comprising epithelial cell-like morphology attached to the surface of the culture vessel; and

(ii) passaging the cells differentiated in step (i) in the presence of a mesenchymal stem cell medium for a time and under conditions sufficient to produce mesenchymal stem cells.

In a second aspect of the present invention there is provided a method for generating mesenchymal stem cells from a population of ESC or iPS, the method comprising:

(i) differentiating a population of ESC or iPS in the presence of an inhibitor of endogenous activin and TGF-β signalling under conditions sufficient to inhibit formation of embryoid bodies (EB); and

(ii) passaging the cells differentiated in step (i) in the presence of a mesenchymal stem cell medium for a time and under conditions sufficient to produce mesenchymal stem cells.

In some embodiments, the present invention provides a method for generating mesenchymal stem cells from a population of ESC or iPS, the method comprising:

(i) differentiating a population of ESC or iPS in the presence of an inhibitor of endogenous activin and TGF-β signalling under conditions that obviate the need to form embryoid bodies (EB); and

(ii) passaging the cells differentiated in step (i) in the presence of a mesenchymal stem cell medium for a time and under conditions sufficient to produce mesenchymal stem cells.

Conditions for inhibiting formation of, or obviating the need to form, EB from human ESC or iPS include, but are not limited to, differentiating the ESC or iPS in a culture vessel having at least one surface at least partially coated with an attachment factor. Thus, in some embodiments of the present invention, the method further comprises differentiating ESC or iPS attached to a surface of a culture vessel by exposing the cells to an inhibitor of endogenous activin and TGF-β signalling to produce a monolayer of cells comprising epithelial cell-like morphology attached to the surface of the culture vessel. Suitable attachment factors include, but are not limited to, fibronectin, laminin collagne IV, enactin, or combinations thereof. A suitable attachment factor is Matrigel™ (BD Biosciences™).

In other embodiments, the ESC or iPS are differentiated in the absence of an attachment factor but become attached to a surface of the culture vessel as a result of the intrinsic property of the surface material.

The term “culture vessel” would be understood by those skilled in the art as meaning any container that can provide a surface on which cells can be culture in accordance with the methods of the present invention, typically under aseptic conditions. Suitable culture vessels include, but are not limited to, tubes, bottles, flasks, plates (including multi-well plates) and bioreactors.

In an embodiment according to the first aspect of the invention, the inhibitor of endogenous activin and TGF-β signalling is SB431542, as hereinbefore described.

In a preferred embodiment of the present invention, the cells induced pluripotent stem cells (iPSs).

The term “epithelial cell-like morphology” as used in this specification is intended to mean differentiated pluripotent cells which possesses epithelial cell like shape and characteristics. A person skilled in the art would appreciate that epithelial cell-like morphology could be determined by physical inspection of cells under a microscope. The pluripotent cells change from colonies with many very small cells on top of each other with almost indistinguishable cell boarders, to larger cells in a monolayer with a typical epithelial morphology (i.e. described as square/cuboidal/cobblestone appearance).

In yet another embodiment of the present invention, prior to differentiation, the pluripotent stem cells are dissociated from mouse embryonic fibroblast (MEF) feeder layer and seeded on Matrigel™-coated flasks in a serum free, feeder layer free media. Serum free, feeder layer free media include mTESR™ medium (Stem Cell Technologies™).

In this specification, the term “serum-free, feeder layer-free media” is intended to mean a cell line derived or cultured in a defined serum-free medium and feeder cells are cells used in co-culture to maintain pluripotent stem cells. Feeder cells usually (but not always) consist of ‘mouse embryonic fibroblasts’ (MEFs), or human fibroblast cells (HFs).

In some embodiments of the present invention, the MEFs are foetal mesenchymal cells obtained from E13.5 foetuses. These cells can proliferate for only a few passages in vitro (primary MEFs) or be immortalized (STO (SIM mouse thioguanine- and ouabain-resistant)-SNL (STO, NEO, LIF) cells).

In some embodiments, when the pluripotent cells are approximately 40% confluent the inhibitor of endogenous activin and TGF-β signalling is added to induce differentiation. In other embodiments of the present invention, the inhibitor of endogenous activin and TGF-β signalling differentiation is added in a medium containing knock-out serum replacement media (KOSR).

When the inhibitor of endogenous activin and TGF-β signalling is SB431542, it is preferably present at a concentration of about 10 μM in, for example, DMEM:F12.

In yet a further embodiment of the present invention, the SB431542+KOSR media is replaced daily, and the cells are differentiated for 1-9 days or when 90% of cells have differentiated to form cells having epithelial-like morphology.

As used herein, the term “mesenchymal stem cell medium” means any culture medium whose substituents, alone or in combination, are capable of supporting the differentiation of the ESC or iPS towards a mesenchymal cell lineage. In some embodiments of the present invention, the mesenchymal stem cell medium is fetal MSC media (fMSC).

In some embodiments, the mesenchymal stem cell medium comprises a high glucose concentration. Examples include, but are not limited to, commercial serum-free or low-serum replacement media (e.g., StemPro™, MesenPro™, MeseCult™) and DMEM-HG. In some embodiments, DMEM-HG comprises:

Calcium Chloride, Anhydrous 200 mg/L
Choline Chloride 4 mg/L
D-Calcium Pantothenate 4 mg/L
D-Glucose, Anhydrous 4500 mg/L
Ferric Nitrate, Nonahydrate 0.1 mg/L
Folic Acid 4 mg/L
Glycine 30 mg/L
HEPES 5958 mg/L
L-Arginine 84 mg/L
L-Cystine, Dihydrochloride 63 mg/L
L-Glutamine 584 mg/L
L-Histidine, Hydrochloride, Monohydrate 42 mg/L
L-Isoleucine 105 mg/L
L-Leucine 105 mg/L
L-Lysine, Hydrochloride 146 mg/L
L-Methionine 30 mg/L
L-Phenylalanine 66 mg/L
L-Serine 42 mg/L
L-Threonine 95 mg/L
L-Tryptophan 16 mg/L
L-Tyrosine, Disodium, Dihydrate 104 mg/L
L-Valine 94 mg/L
Magnesium Sulfate, Anhydrous 97 mg/L

In some embodiments, the mesenchymal stem cell medium is supplemented with 10% fetal bovine serum, 20% fetal bovine serum, autologous or allogeneic human serum, mammalian (e.g., human) platelet lysate, or combinations thereof.

It will be understood by those skilled in the art that the composition of DMEM-HG, as hereinbefore described, is but one example that may be used in accordance with the methods of the present invention and that changes can be made by adding or removing substituents and/or altering the concentration of the substituents (including serum supplements) without departing from the ability of the culture medium to support the differentiation of ESC or iPS into mesenchymal stem cells.

In some embodiments of the present invention, human embryonic stem cells (hESCs) colonies are cultured feeder-free until confluent, and then EMT-like state induced by adding SB431542, an inhibitor of TGF-β receptor kinases, for 10 days prior to passaging into fetal MSC media (fMSC). Specifically, the hESC line, MEL1, produced cells that were plastic adherent with a characteristic MSC-like morphology. ES-derived MSC expressed a typical MSC surface immunophenotype (CD73⁺, CD105⁺, CD90⁺, CD44⁺, CD29⁺, CD45⁻, CD31⁻, CD11b⁻). These ES-MSC expressed low HLA-ABC and no HLA-DR indicating they may be immune tolerable in vivo similar to MSC. Osteogenic and chondrogenic differentiation was induced in vitro in all three MSC populations, although adipogenic differentiation was limited, as has been observed for primitive fetal MSC. Differentiation of MEL1 hESC resulted in loss of the pluripotency marker Oct4, and increased vimentin and collagen I expression.

In some embodiments, the EB and SB431542 inhibitor differentiation methods can be applied to the human iPSC line ES4CL1 to produce MSC-like cells with characteristic fibroblast-like morphology and an immunophenotype similar to MSC.

The present invention also contemplates a mesenchymal cell or mesenchymal cell population generated according to the methods of the invention. Accordingly, in a third aspect of the present invention there is provided a mesenchymal cell or population of mesenchymal cells generated by performing the method according to the first and/or second aspects of the present invention.

The present invention also contemplates a pharmaceutical composition comprising a mesenchymal cell or population of mesenchymal cells generated by performing the method according to the first and/or second aspects of the present invention together with a pharmaceutically suitable carrier or excipient.

An exemplary carrier is an aqueous pH buffered solution. Examples of pharmaceutically acceptable carriers include, but are not limited to, saline, solvents, dispersion media, cell culture media, aqueous buffers such as phosphate, citrate, and other organic acids; antioxidants including ascorbic acid; low molecular weight (less than about 10 residues) polypeptides; proteins, such as serum albumin, gelatine, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEENT™, polyethylene glycol (PEG) and PLURONICS™.

Additional suitable pharmaceutically acceptable carriers or excipients will be apparent to the skilled artisan and/or described in U.S. Pharmacopeia, or the European Pharmacopeia or “Remington's Pharmaceutical Sciences” by E. W. Martin. Pharmaceutical carriers suitable for use in a composition of the present invention should not be toxic to a cell of the present invention. The pharmaceutical composition of the invention can also contain an additive to enhance, control, or otherwise direct the intended therapeutic effect of the cells comprising said pharmaceutical composition, and/or auxiliary substances or pharmaceutically acceptable substances, such as minor amounts of pH buffering agents, tensioactives, co-solvents, preservatives, etc. A pharmaceutical composition of the invention can additionally or alternatively comprise a metal chelating agent and/or an amino acid such as aspartic acid, glutamic acid, etc. A pharmaceutical composition of the present invention can also comprise an agent to facilitate storage of the composition and cells therein, e.g., a cryopreservative. Illustrative, non limiting, examples of carriers for the administration of the cells contained in the pharmaceutical composition of the invention include, for example, a sterile saline solution (0.9% NaCl), PBS.

A pharmaceutical composition of the present invention can also comprise a bioactive agent (such as, for example, a growth factor) to reduce or prevent cell death and/or to enhance cell survival and/or to enhance cell differentiation and/or proliferation.

The pharmaceutical composition of the invention will contain a prophylactically or therapeutically effective amount of the cells of the invention, preferably in a substantially purified form, together with the suitable carrier or excipient. In one embodiment, the pharmaceutical composition comprises between about 1×10⁵ to about 1×10¹³ cells, e.g., between about 2×10⁵ to about 8×10¹² cells.

The pharmaceutical composition of the invention is formulated according to the chosen form of administration. The formulation should suit the mode of administration. In a particular embodiment, the pharmaceutical composition is prepared in a liquid dosage form, e.g., as a suspension, to be injected into a subject in need of treatment. Illustrative, non limiting examples, include formulating the cells of the invention in a sterile suspension with a pharmaceutically acceptable carrier or excipient, such as saline solution, phosphate buffered saline solution (PBS), or any other suitable pharmaceutically acceptable carrier, for parenteral administration to a subject, e.g., a human being, e.g., intravenously, intraperitonealy, subcutaneously, etc.

A person skilled in the art will appreciate that the mesenchymal cell or population of mesenchymal cells generated by performing the method according to the present invention will be useful in cell replacement therapies which typically use human bone marrow MSC for the repair of congenital bone diseases such as osteogenesis imperfecta or non-union bone fractures. Other conditions that may be treated or prevented by the use of a mesenchymal cell or a population of mesenchymal cells generated by the method according to the present invention include, but are not limited to, cardiac repair (e.g., post-myocardial infarction), cartilage repair, osteoarthritis, haematological conditions (e.g., graft-versus-host disease, co-transplantation with cord blood and/or bone marrow and/or haematopoietic stem cells), inflammatory bowel disease, sepsis, stroke, multiple sclerosis, renal impairment, ex vivo or in vivo regeneration of cartilage and/or bone and for facilitating drug or gene delivery in the treatment of cancer or genetic disorders.

In another aspect of the present invention, there is provided a tissue matrix comprising a mesenchymal cell or population of mesenchymal cells generated by a method of the present invention, as herein described.

As used hereinafter, the term “tissue matrix” typically refers to a material scaffold of interconnected open porosity that is, preferably, biocompatible and, preferably, elicits minimal inflammation or an immune response when incorporated into a living being (e.g., humans or animal). The tissue matrices according to some embodiments of the present invention are applied to the formation and delivery of tissue healing scaffolds to damaged or degenerated joint or soft tissue. Biological remodelling of the matrix scaffold depends, in part, upon the ability of mesenchymal stem cells to migrate into the matrix and regenerate a biocompatible tissue. Accordingly, the structural and biochemical characteristics of the matrix may be further optimized to promote specific chemical, nutritional or tissue migration. Mechanical and biological performances of some tissue matrix scaffolds are well known to those familiar with the art.

As used herein, the term “tissue matrix material” refers to porous and nonporous polymeric compounds that are cytocompatible, biologically inert, non-inflammatory, nontoxic and generate minimal immune reaction when incorporated into a living being (e.g., humans).

The tissue matrix may comprise material that is non-degradable and/or degradable. A “degradable” tissue matrix is typically made of a material that can be degraded and absorbed in situ in a living being such as human.

In some embodiments, the tissue matrix will either permanently or temporarily augment the damaged and degenerated tissues to restore functionality. The matrix should also function as a porous scaffold possessing physicochemical properties suitable for use in the repair and regeneration of musculoskeletal soft tissues (tendons, cartilage and fibrotic scar tissue). The tissue matrix material can be naturally derived or synthetic and may be formed in situ in the presence of cells and tissues. The matrices also typically satisfy the requirements for cellular tissue repair. This requires precise control of porosity and internal pore architecture to ensure blood flow and adequate diffusion of nutrients and interstitial fluid, optimize cell migration, growth and differentiation and maximize the mechanical function of the matrices and the regenerated tissues.

Examples of naturally-derived tissue matrix material include, but are not limited to, fibrin, collagen (e.g., Type I, II, and III collagen), fibronectin, laminin, polysaccharides (e.g., chitosan), polycarbohydrates (e.g., porteoglycans and glycosaminoglycans), cellulose compounds (e.g., methyl cellulose, carboxymethyl cellulose, and hydroxy-propylmethyl cellulose) and combinations thereof. Examples of synthetic compositions that satisfy these requirements include, but are not limited to, aliphatic polyesters (e.g., polylactides (PLA), polycaprolactone (PCL) and polyglycolic acid (PGA)), polyglycols (e.g., polyethylene glycol (PEG), polymethylene glycol, polytrimethylene. glycols), polyvinyl-pyrrolidones, polyanhydrides, polyethylene oxide (PEO), polyvinyl alcohols (PVA), poly(thyloxazoline) (PEOX), polyoxyethylene and combinations and derivatives thereof. The tissue matrix material may be obtained autologously or supplemented endogenously with host body fluids to increase their biocompatibility with host tissues.

In some embodiments, the tissue matrix material is fibrin. The formation of fibrin mimics the final stage of the natural clotting mechanism. Fibrin formation is initiated following activation of fibrinogen by a fibronogen activating agent such as thrombin and reduction of fibrinogen into fibrinopepetides. The fibrinopeptides spontaneously react and polymerize into fibrin. Fibrinogen can be isolated from autologous (i.e., from the patient to be treated), heterologous (i.e., from other human, pooled human supply, or non-human sources) tissues or recombinant sources. Fibrinogen can be provided in fresh or frozen solutions.

A tissue matrix can be processed to remove any native cells and other antigens and cellular debris to form a substantially decellularized tissue matrix, and, optionally, treated to inhibit generation of immunological sites, particularly where the tissue matrix is xenogeneic or allogeneic. Optionally, this tissue matrix can then be treated with attachment factors (e.g., cellular adhesion factors) as herein described to enhance attachment of mesenchymal stem cells to the matrix during the process of repopulating the tissue matrix with such cells.

Depending on the type of transplant intended, if the recipient is human, the initial transplant tissue or organ may be of non-human origin. These tissues or organs may be obtained from animals. The tissues or organs are typically handled in a sterile manner, and any further dissection of the tissue or organs is carried out under aseptic conditions. After collection and dissection, this tissue may be sterilized by incubating it in a sterile buffered nutrient solution containing antimicrobial agents. The sterilized transplant tissue may then be cryopreserved for further processing at a later time or may immediately be further processed according to the next steps of this process including a later cryopreservation of the tissue matrix or other tissue products of the process.

In some embodiments, particularly where the tissue matrix has been naturally derived (e.g., isolated from an animal or human being), the tissue matrix is first decellularized. Several means of decellularizing a tissue or organ are known, including physical, chemical, and biochemical methods (see, e.g. U.S. Pat. No. 5,192,312), incorporated herein by reference. It is preferable that the decellularization technique employed should not result in gross disruption of the anatomy of the tissue or organ substantially alter the biomechanical properties of their structural elements. The treatment of the tissue to produce a decellularized tissue matrix should also preferably not leave a cytotoxic environment that mitigates against subsequent repopulation of the matrix with the mesenchymal stem cells generated by a method of the present invention that are allogeneic or autologous to the recipient. Cells and tissues that are allogeneic to the recipient are those that originate with or are derived from a donor of the same species as the recipient. Autologous cells or tissues are those that originate with or are derived from the recipient.

Physical forces can also be used to decellularize a tissue matrix. For example, vapor phase freezing (slow rate of temperature decline) of intact heart valves can reduce the cellularity of the heart valve leaflets as compared to liquid phase freezing (rapid). Colloid-forming materials may be added during freeze-thaw cycles to alter ice formation patterns in the tissue. Polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. This allows a measure of decellularization while providing the tissue matrix with some protection from ice damage.

Alternatively, various enzymatic or other chemical treatments to eliminate viable native cells from tissues or organs may be used, although care must generally be taken to minimise or avoid extended exposure of the tissue matrix to proteases such as trypsin, as metrix protein such as type I collagen molecule is sensitive to a variety of proteases, including trypsin.

Combinations of different classes of detergents, for example, a nonionic detergent, Triton X-100, and an anionic detergent, sodium dodecyl sulfate, may also disrupt cell membranes and aid in the removal of cellular debris from a tissue matrix.

The decellularization of the transplant tissue is preferably accomplished by the administration of a solution effective to lyse native cells present within the tissue matrix.

It is preferred that the decellularization treatment of the tissue matrix also limits the generation of new immunological sites. While collagen is typically substantially non-immunogenic, partial enzymatic degradation of collagen may lead to heightened immunogenicity. Accordingly, a preferable step of this process includes treatment of the tissue with enzymes, such as nucleases, effective to inhibit cellular metabolism, protein production and cell division without degrading the underlying collagen matrix. Nucleases that can be used for digestion of native cell DNA and RNA include both exonucleases and endonucleases. A wide variety of which are suitable for use in this step of the process and are commercially available.

Other enzymatic digestions may be suitable for use herein, for example, enzymes that will disrupt the function of native cells in a tissue matrix. For example, phospholipase, particularly phospholipases A or C, in a buffered solution, may be used to inhibit cellular function by disrupting cellular membranes of endogenous cells. Preferably, the enzyme employed should not have a detrimental effect on the tissue matrix protein. The enzymes suitable for use may also be selected with respect to inhibition of cellular integrity, and also include enzymes which may interfere with cellular protein production. The pH of the vehicle, as well as the composition of the vehicle, will also be adjusted with respect to the pH activity profile of the enzyme chosen for use. Moreover, the temperature applied during application of the enzyme to the tissue should be adjusted in order to optimize enzymatic activity.

Following decellularization, the tissue matrix may be washed to assure removal of cell debris which may include cellular protein, cellular lipids, and cellular nucleic acid, as well as any extracellular debris such as extracellular soluble proteins, lipids and proteoglycans. Removal of this cellular and extracellular debris reduces the likelihood of the tissue matrix eliciting an adverse immune response from the recipient upon implant. For example, the tissue may be incubated in a balanced salt solution such as Hanks' Balanced Salt Solution (HBSS). The composition of the balanced salt solution wash, and the conditions under which it is applied to the transplant tissue matrix may be selected to diminish or eliminate the activity of the nuclease or other enzyme utilized during the decellularization process. Optionally, an antibacterial, an antifungal or a sterilant or a combination thereof, may also be included in the balanced salt wash solution to protect the tissue matrix from contamination with environmental pathogens.

The tissue matrix, whether or not having been cryopreserved, may be next treated to enhance the attachment (adhesion) and inward migration of the mesenchymal stem cells, in vitro, which will be used to repopulate the transplant tissue.

The extent of attachment may be increased by the addition of serum (human or fetal bovine, maximal binding with 1% serum) and by purified fibronectin to the culture medium. Each of the two homologous subunits of fibronectin has two cell recognition regions, the most important of which has the Arg-Gly-Asp (RGD) sequence. A second site, binding glycosaminoglycans, acts synergistically and appears to stabilize the fibronectin-cell interactions mediated by the RGD sequence. Heparin sulfate along with chondroitin sulfate are two glycosaminoglycans identified on cell surfaces. Heparin sulfate is linked to core proteins (syndecan or hyaluronectin) which can either be integral or membrane spanning. Cellular binding sites for extracellular matrix glycoproteins are called integrins and these can mediate tight binding of cells to the adhesion factors. Each attachment factor typically comprises a specialized integrin, although a single integrin may bind to several extracellular matrix factors.

Delivery for any of the described tissue matrices can be achieved by percutaneous injection into the tissue or joint under direct visualization or with fluoroscopic control, or by direct injection into the tissue or joint in an open, mini-open or endoscopic procedure. The tissue matrix may be administered or combined with one or more biological additives to reduce pain and/or enhance joint and tissue healing. As used herein, the term “biological additives” includes: anesthetics and/or analgesics (e.g., lidocaine and bupivicaine); proteoglycans (e.g., sGAG, aggrecan, chondrotin sulfate, deratin sulfate, versican, decorin, fibronectin and biglycan); hyaluronic acid and salts and derivatives thereof; pH modifiers and buffering agents; anti-oxidants (e.g., superoxide dismutase, and melatonin); protease inhibitors (e.g., TIMPtypes I, II, III); cell differentiation and growth factors that promote healing and tissue regeneration (e.g., TGFβ, PDGF, BMP-2,6,7, LMP-1, and CSF); biologically active amino acids, peptides, and derivatives thereof (e.g., fibroblast attachment peptides such as Arg-Gly-Asp, (RGD), Arg-Gly-Asp-Ser (RGDS), Gly-Arg-Gly-Asp-Ser (GRGDS), P-15 and fibroblast migration peptides such as Met-Ser-Phe (MSF) and Ile-Gly-Asp (IGD), and Gly-Asx-Asp (GBD)); anti-inflammatory agents (e.g., erythropoietin-corticosteroid); antibiotics; antifungals; antiparasitics; histamines; antihistamines; anticoagulants; vasoconstrictors, vasodilators; vitamins; cellular nutrients (e.g., glucose and other sugars); gene therapy reagents (e.g., viral and non-viral vectors); salicylic acid and derivatives of salicylic acid (e.g., acetylsalicylic acid).

In addition to mesenchymal stem cells, the tissue matrix according to the present invention may also comprise or be administered with one or more cellular and biological additives.

As used herein, the term “cellular additives” includes any kind of cells that could further assist in the repair of the damaged or degenerated joint and/or tissue. Appropriate cells include, but are not limited to, autologous fibroblasts from dermal tissue, oral tissue, or mucosal tissue; autologous chondrocytes or fibroblasts from tendons, ligaments or articular cartilage sources; allogenic juvenile or embryonic chondrocytes; embryonic stem cells; and genetically altered cells. Precursor cells of chondrocytes, differentiated from stem cells, can also be used in place of the chondrocytes. As described herein, the term “chondrocytes” includes chondrocyte precursor cells.

In some embodiments, the tissue matrix is premixed with a cellular additive prior to injection. In other embodiments, the tissue matrix is mixed with a cellular additive during the injection. In other embodiments, the tissue matrix is injected first, followed with an injection of a cellular additive. In other embodiments, a cellular additive is injected first, followed with an injection of the tissue matrix. In all cases, the tissue matrix functions as a matrix scaffold for cell proliferation, migration and matrix formation at or around the injection site. Typically, the injection of cells is performed under physiologic conditions to maintain cell viability.

The present invention also contemplates a mesenchymal cell or population of mesenchymal cells generated by performing the method according to one aspect of the invention, or a pharmaceutical composition according to another aspect of the present invention or a tissue matrix according to another aspect of the present invention, for use in human therapy or for veterinary use.

In order that the present invention may be more clearly understood, preferred examples thereof will now be described with reference to the following non-limiting examples.

### EXAMPLES

### Materials

SB431542 is an inhibitor of the TGF-β1 activin receptor-like kinases (ALKs). It is a selective and potent inhibitor of ALK-4, -5 and -7. SB431542 inhibits endogenous activin and TGF-β signaling without affecting more divergent BMP signaling utilizing ALK-1, -2, -3, and -6 (Inman et al. (2002) Mol. Pharmacol. 62:65-74; Laping et al. (2002) Mol. Pharmacol 62:58-64).

### Methods

### SB431542 Differentiation Method:

Day −4 to −1: hESC/iPSC were dissociated from mouse embryonic fibroblast (MEF) feeder layer and seeded on either un-coated flasks (i.e., which allow for adherence (attachment) of the pluripotent cells to the surface) or matrigel-coated flasks in mTESR (defined pluripotent stem cell media, Stem Cell Technologies; i.e. serum free, feeder layer free).

Day 1: when cell colonies were approximately 40% confluent, 10 uM SB431542 inhibitor (Sigma Aldrich) was added in DMEM:F12+20% knock out serum replacement (KOSR, Invitrogen) media.

Day 2-9: 20% KOSR+SB431542 inhibitor media replaced daily.

Day 7-10: when 90%+ cells have differentiated from hESC colonies to a monolayer with epithelial-like morphology, cells were passaged using TrypleSelect. Single cells were then seeded at 3×10⁴-1.5×10⁵ cells/cm² in fetal MSC medium (DMEM-HG+10% MSC qualified FBS), then passage when nearly confluent as for MSC.

The embryoid body (EB)-derived MSC were also produced for comparison with MSC derived by a method according to one embodiment of the present invention. Briefly, confluent iPS and ESC colonies were cultured on mouse embryonic fibroblasts (mef) (approx. 12,000 cells/cm²) in 20% KOSR medium supplemented with basic fibroblast growth factor (bFGF; 10 ng/ml for ESC and 100 ng/ml for iPSC). Colonies were detached from the flask using a cell scraper and cultured 1:1 as EB for approximately 10 days in 10 cm non-tissue culture treated dishes. EB were then transferred to a standard tissue culture flask containing fMSC medium to adhere to the flask. Differentiated cells grew outwards from the centre of the EB and formed a heterogeneous cell layer. After approximately one week, the undifferentiated cells in the centre of the colony were aspirated and the differentiated outgrowth cells were further cultured in fMSC medium as per standard fMSC culture at a density of 40,000 cells/cm² at the first mesenchymal passage (mp0) and then at 5,000-10,000 cells/cm² for all subsequent passages (see Hwang et al., Tissue Engineering, 2006, 12(6):1381-1392 and Xu et al., Stem Cells, 2004, 22:972-980). These cells were designated as ES-MSC (EB) or iPS-MSC (EB). Where reference is made to ES-MSC or iPS-MSC (or where reference to “EB” is absent), this is to be understood as a reference to MSC derived by a method according to the present invention (e.g., ES-MSC (inhibitor) or iPS-MSC (inhibitor)).

### Results

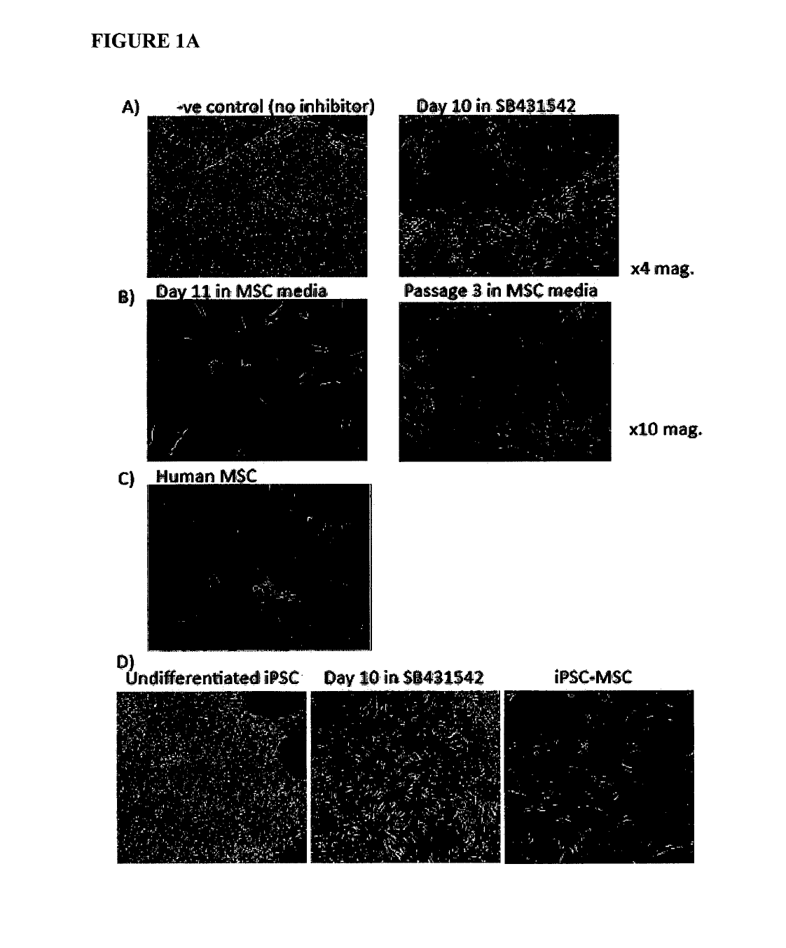

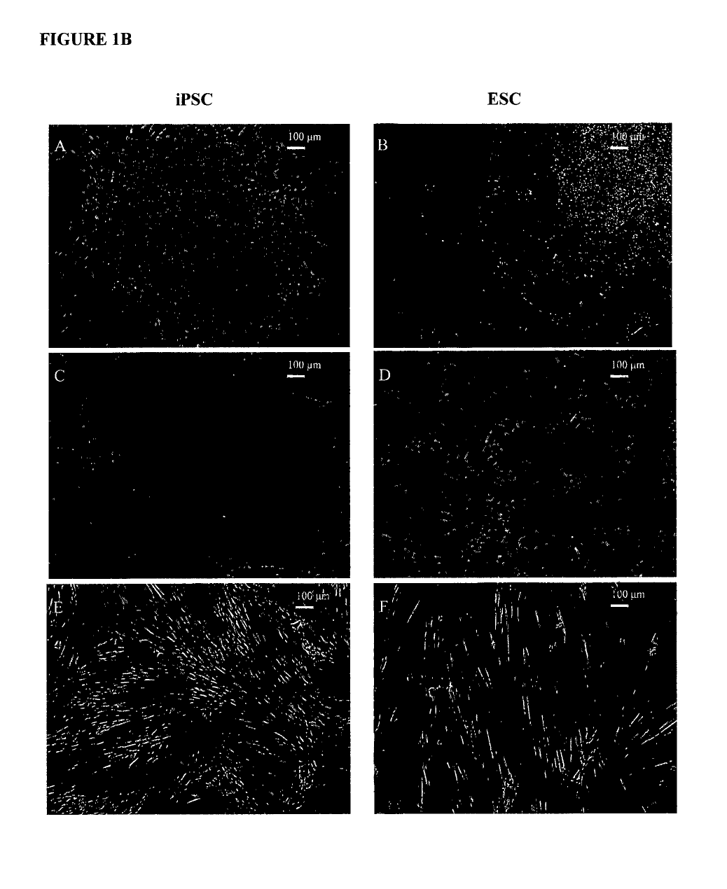

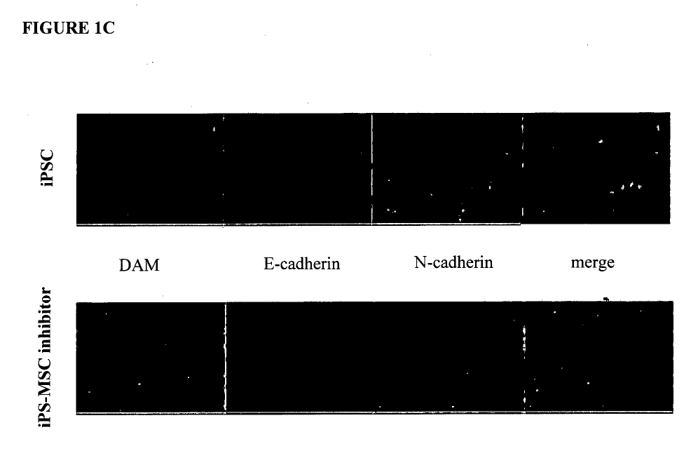

The SB431542 inhibitor differentiation methods applied to the hESC line, MEL1, produced cells that were plastic adherent with a characteristic MSC-like morphology (referred to as ES-MSC; FIG. 1). The resulting cells did not require attachment factor such as gelatin, fibronectin or matrigel, nor did they require a feeder layer to support growth, unlike undifferentiated hESC.

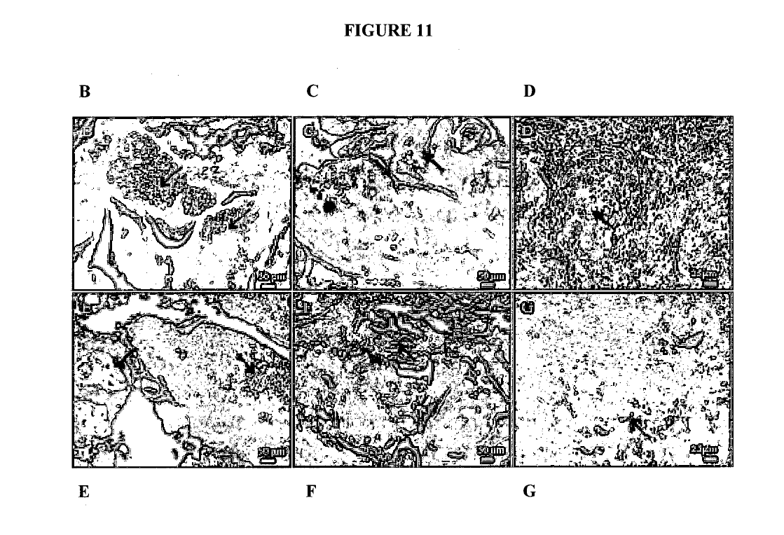

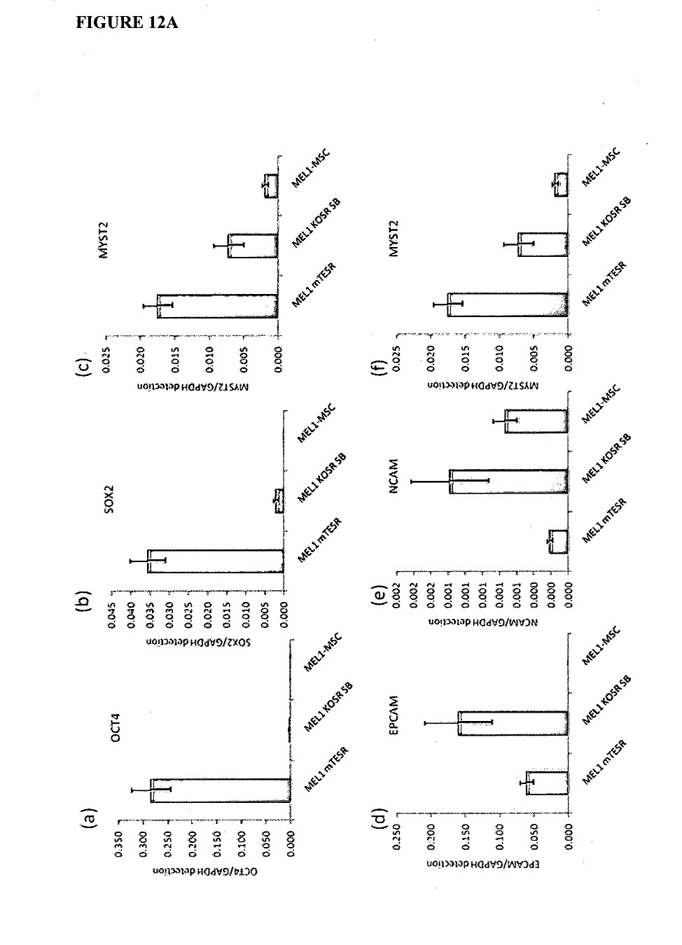

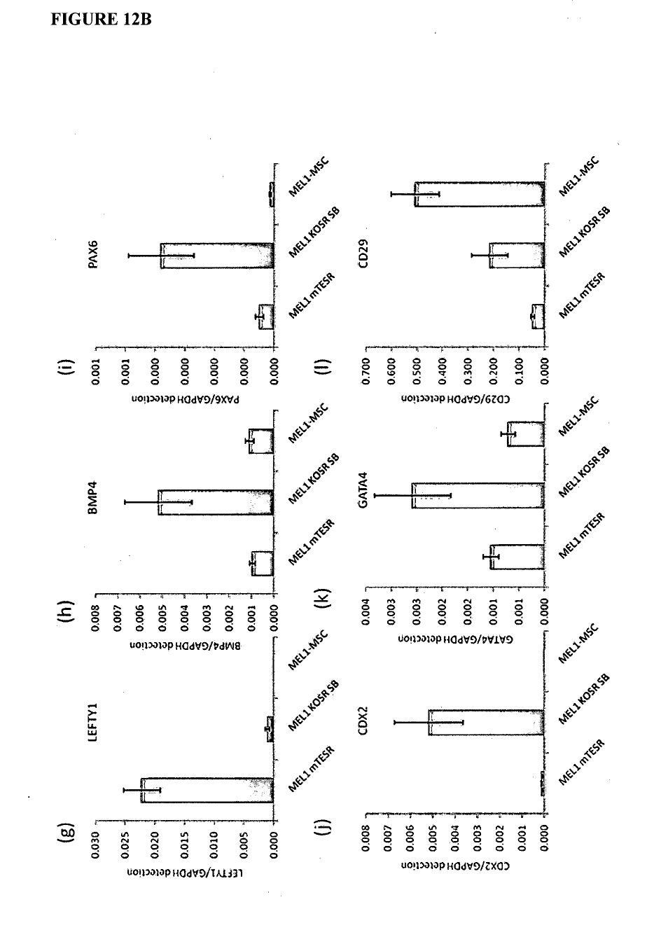

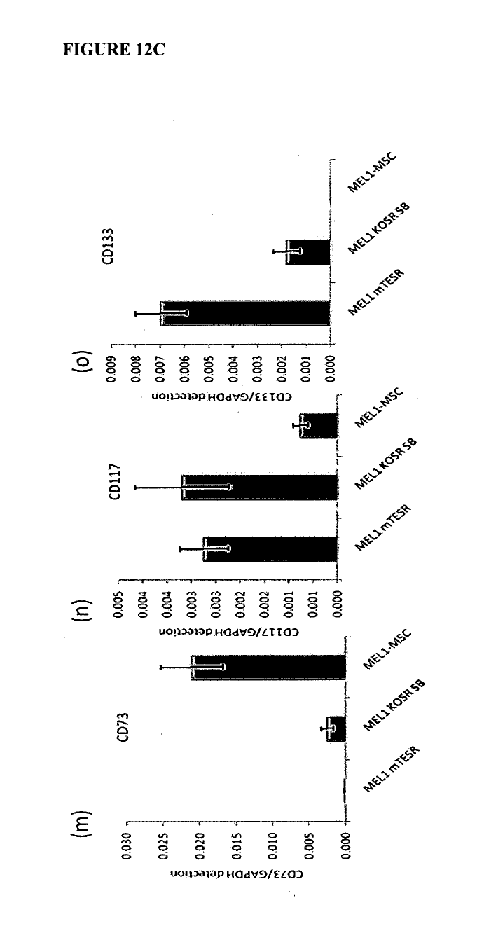

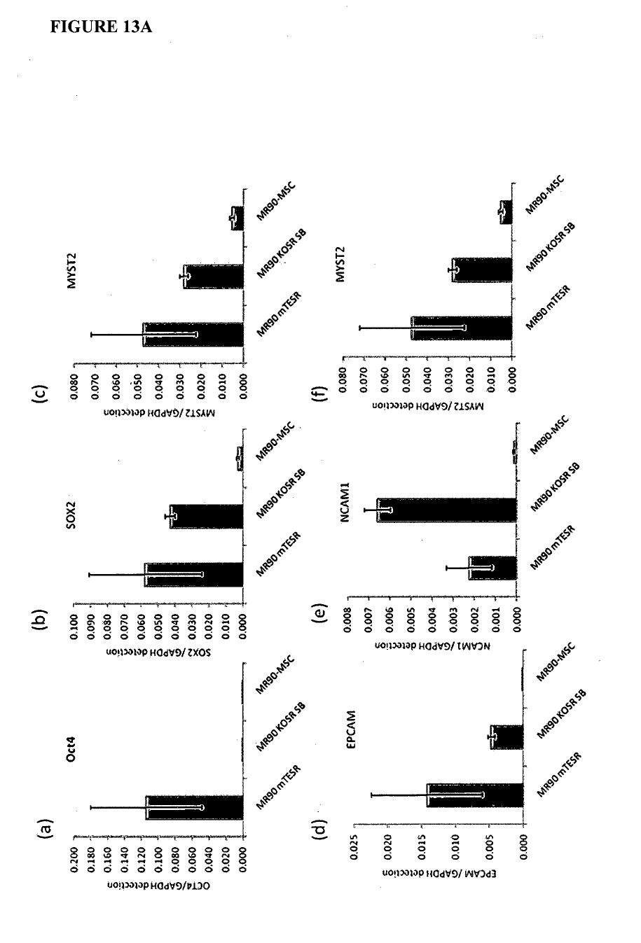

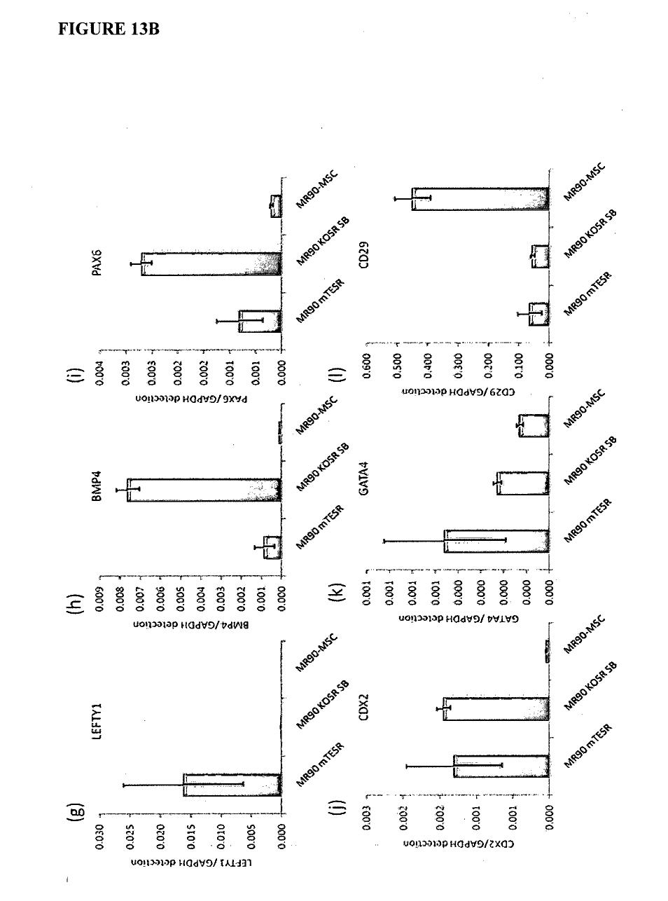

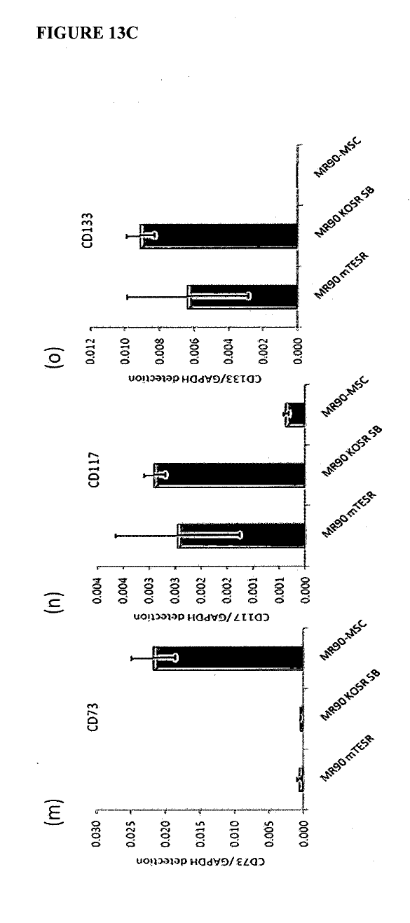

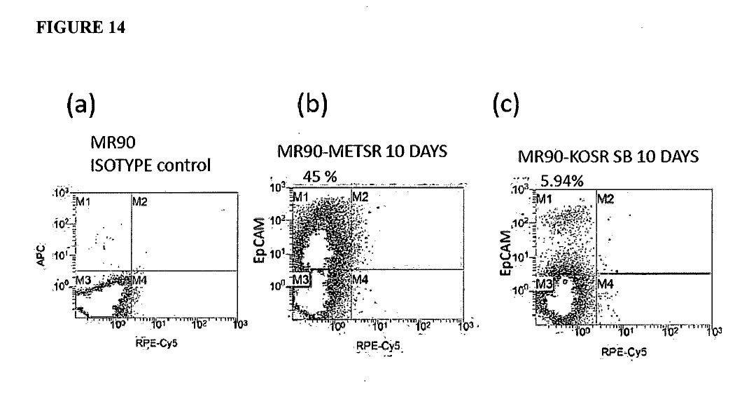

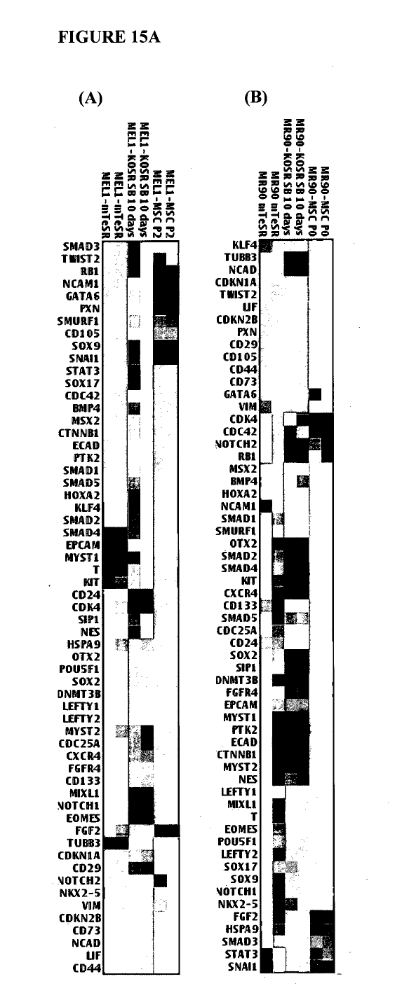

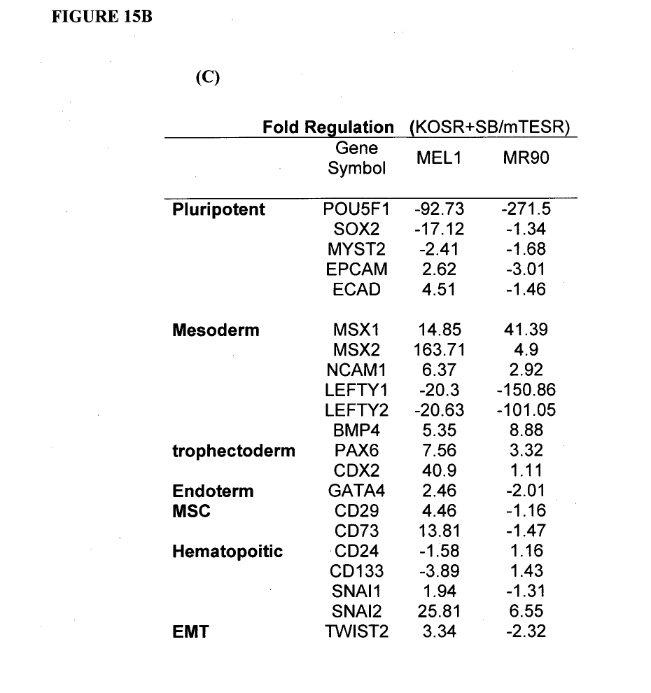

Differentiation of iPSC/ESC was also induced in the presence of 10 μM SB431542 in Matrigel-coated dishes in serum- and feeder-free culture conditions (KOSR medium) for 10 days to generate a uniform monolayer of cells comprising epithelial-like morphology. To address the progression of iPSC-derived MSC, qRT-PCR and flow cytometry were used to identify the iPSC directed toward mesoderm-derived MSC. Both of the MEL1 and MR90 cells cultured with SB431542, the qRT-PCR data demonstrated that the relative mRNA levels of puripotent genes, such as Pou5F (Oct4), SOX2, MYST2 were decreased compared with those of the undifferentiated ESCs/iPSCs cultured in mTeSR medium (FIGS. 11-13). The heat map data showed that SB431542-induced MSC cells cultured in MSC medium perform strong MSC marker expression (CD29, CD73; see FIG. 15). As shown in these data, the MEL1 and MR90 have similar effect in the loss of pluripotent gene expression and an increase of mesoderm and ectoderm genes expression. SB431542 also enhanced MEL1 differentiation into mesoderm (MSX1, MSX2, SOX9, NCAM1, BMP4), ectoderm (PAX6), trophectoderm (CDX2), endoderm (GATA4) and MSC cells (CD73, CD29). Interestingly, the SB431542 induced MR90 differentiate into mesoderm (MSX1, MSX2, NCAM1, BMP4), ectoderm (PAX6) and trophectoderm (CDX2). These data indicate that ESC (MEL1) and iPS (MR90) have similar differentiation status's (mesoderm and ectoderm) over 10 days in the presence of SB431542.

Immunofluorescence revealed that undifferentiated iPSC (induced pluripotent stem cells; iPS) expressed E-cadherin throughout the colony, whereas N-cadherin expression was limited to the periphery of the cell colony where spontaneously differentiating cells were localized (FIG. 1C).

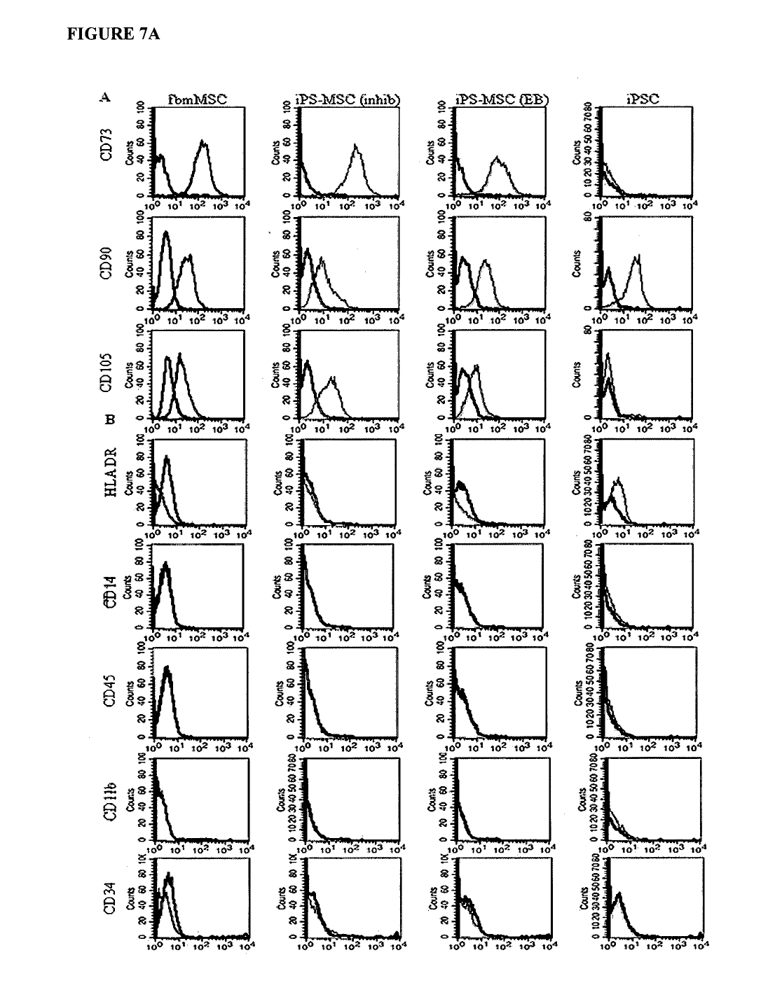

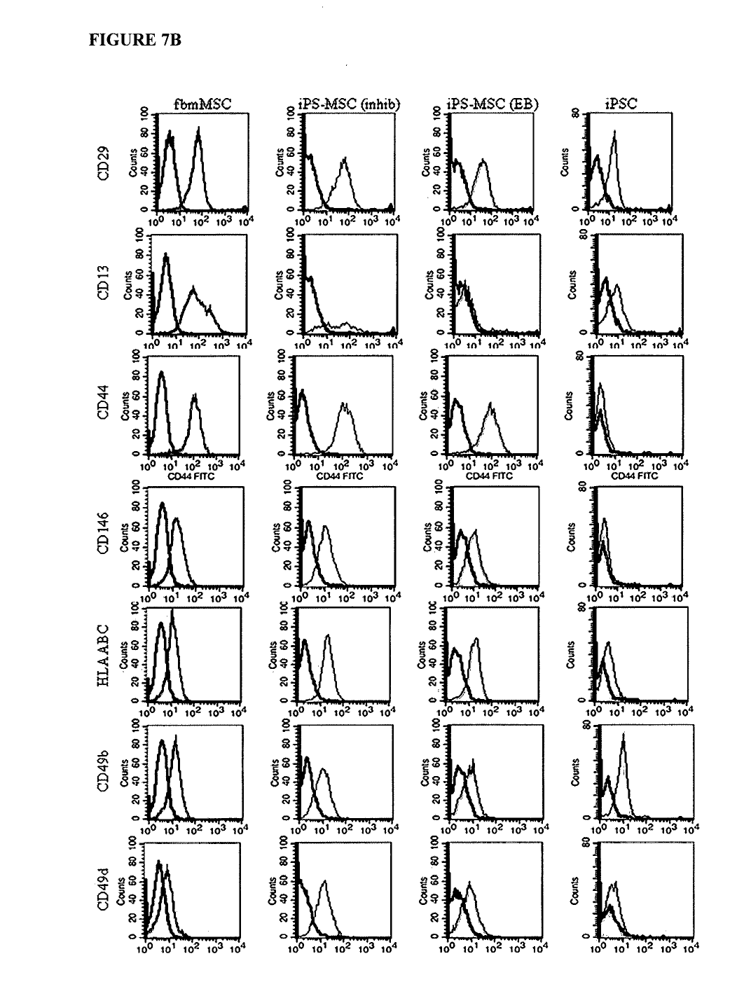

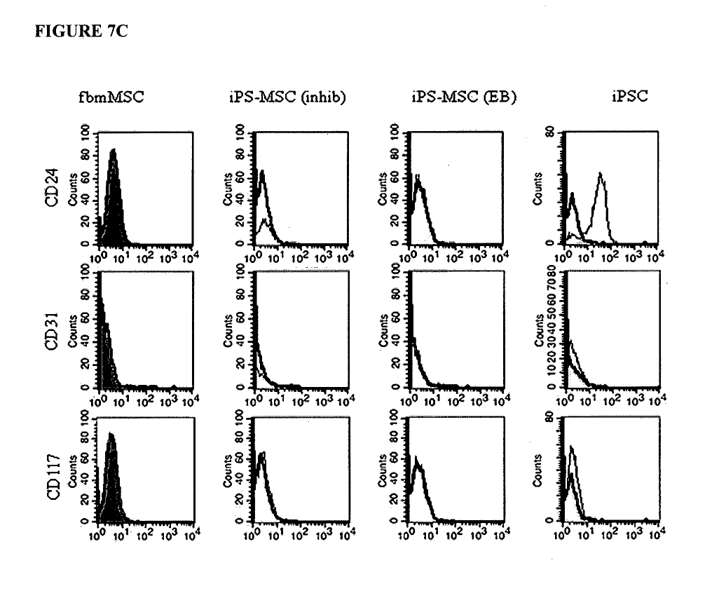

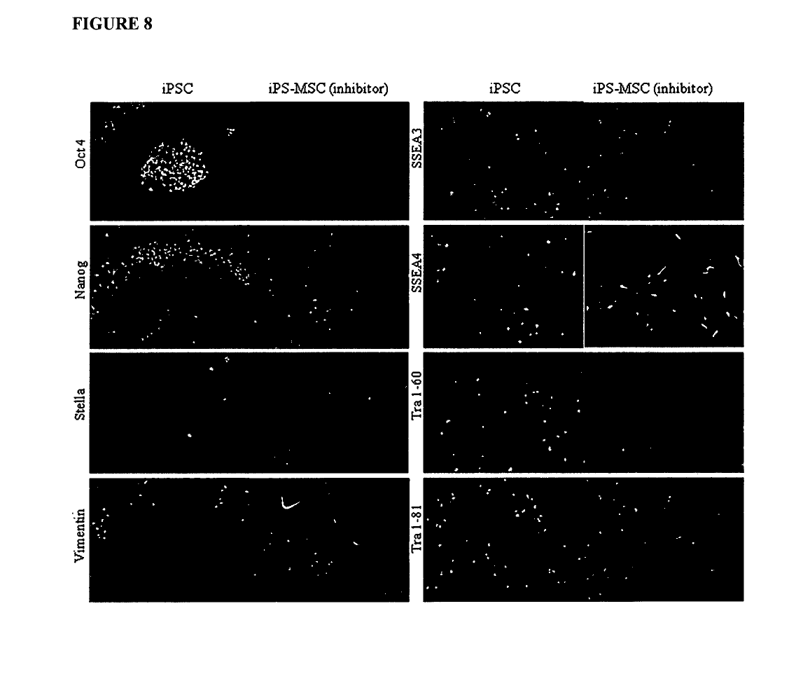

iPS-MSC also expressed positive MSC markers (CD73, CD90 and CD105) at similar levels to fMSC (FIGS. 7A-7C). All MSC samples lacked expression of macrophage and monocyte markers (CD11b and CD14), human leukocyte marker (HLA-DR) and broad hematopoietic markers (CD45) and broad hematopoietic progenitor marker (CD34). iPS-MSC derived by culturing in the presence of SB431542 and fMSC also expressed other markers common to MSC, including CD29, CD13, CD44 and CD146. Also consistent with criteria for defining MSC, iPS-MSC expressed low levels of HLA-ABC and lacked expression of HLA-DR. By contrast, fMSC and iPS-MSC derived by culturing in the presence of SB431542 and through formation of embryoid bodies (EB) lacked expression of the ESC and pericyte marker, CD24, which was expressed positively by the iPSC sample as expected. The endothelial marker, CD31, and the primitive haematopoietic and progenitor cell marker, CD117, were also not expressed by fMSC, iPS-MSC (SB431542), iPS-MSC (EB) and iPS.

iPS-MSC (inhibitor) cells (i.e., iPS differentiated in the presence of SB431542) at mesenchymal passage 2 (mp2) showed no expression of E-cadherin, but expressed N-cadherin outside the nucleus of all cells viewed, as seen in definitive EMT. Thus, with differentiation, the classic E-cadherin to N-cadherin switch was observed, as in definitive EMT.

EPCAM was also present in the pluripotent stem cells (FIG. 14). The EPCAM-(CD326-) cell population have been identified as the precursors of the mesodermal cell lineage. These cells can be further differentiated into mesenchymal stem cell lineage, like MSC. The iPSC MR90 cells were cultured in mTeSR and KOSR condition medium with SB431542 for 10 days, and then subcultured into MSC medium to differentiate cells into MSC. The population of EPCAM+ MR90 cell was decreased from 45% to 8.14% in MTESR medium with SB431542. The population of EPCAM+ MR90 cell was decreased from 22.84% to 5.94% in the KOSR medium with SB431542.

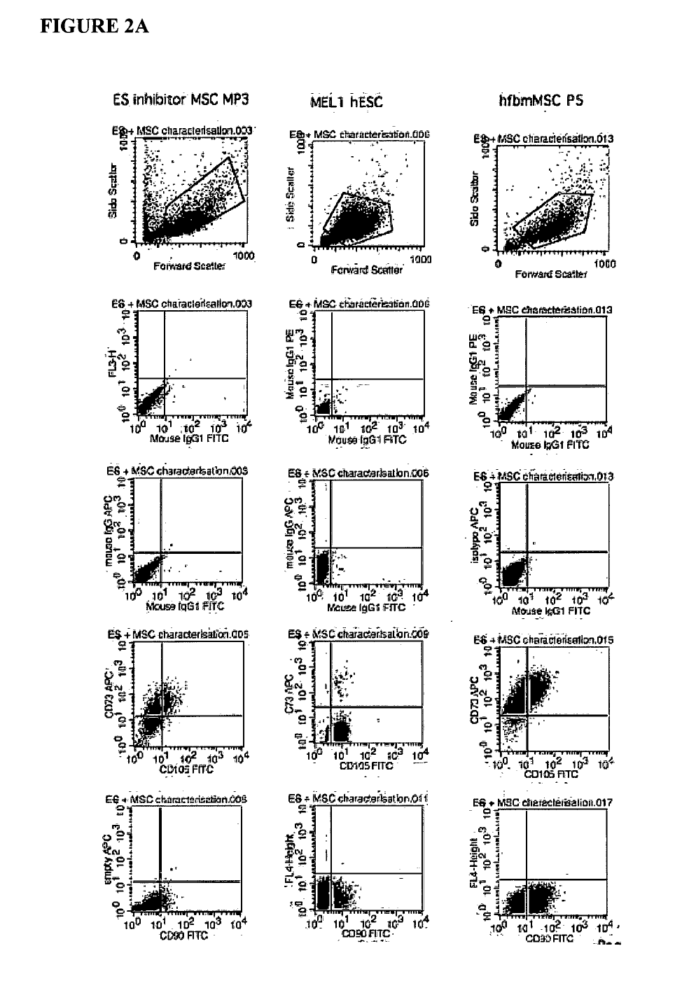

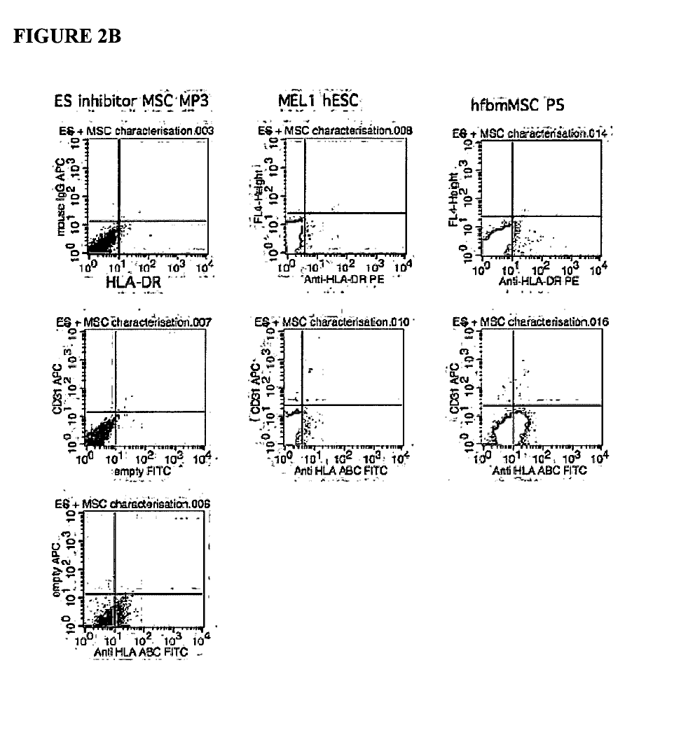

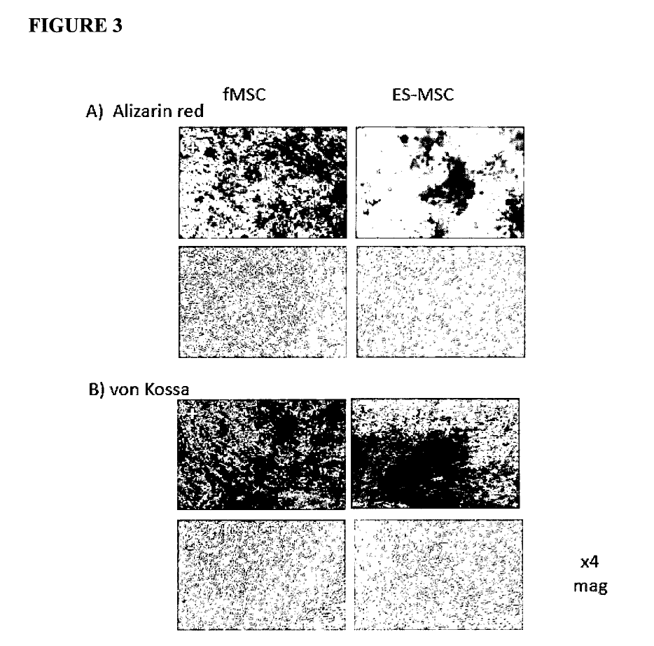

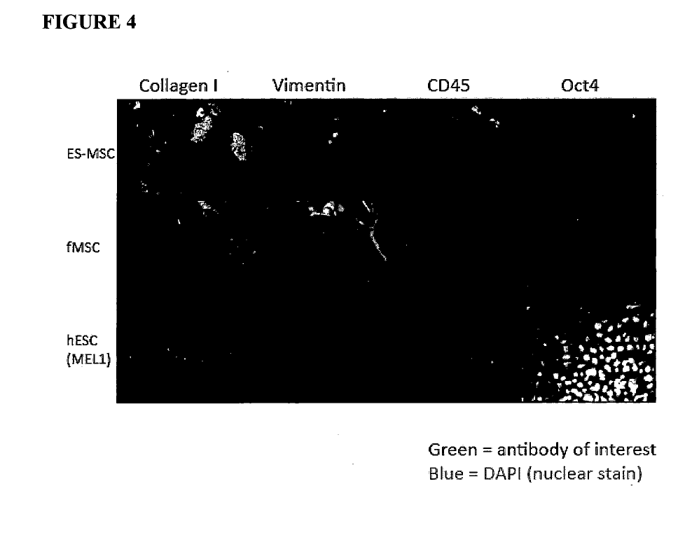

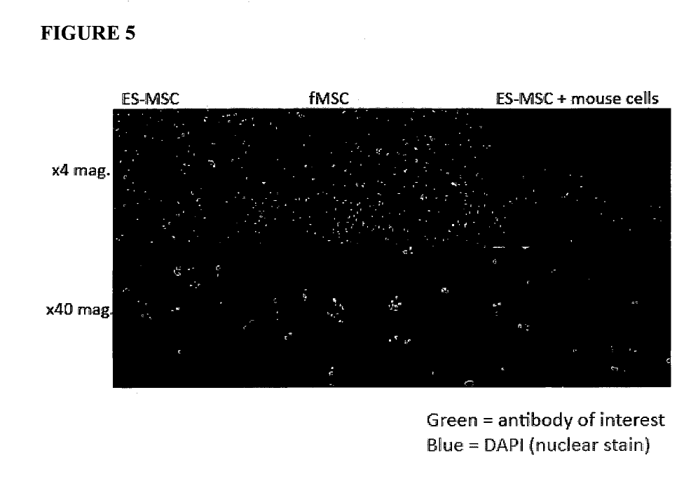

ES-MSC expressed a typical MSC surface immunophenotype: CD73⁺, CD105⁺, CD90⁺, CD45⁻ and CD31⁻ (FIG. 2). The ES-MSC expressed low HLA-ABC and no HLA-DR, indicating they may be immune tolerable in vivo, similar to MSC (FIG. 2). Osteogenic and chondrogenic differentiation was induced in vitro in ES-MSC, although adipogenic differentiation was limited, as has been observed for primitive fetal MSC (FIG. 3). Differentiation of MEL1 hESC resulted in loss of the pluripotency markers including Oct4, increased mesodermal marker expression (vimentin⁺ and collagen I₊) and no hematopoietic lineage differentiation (CD45⁻; FIG. 4). To confirm the ES-MSC were not contaminating mouse embryonic fibroblasts, cells were stained with a human-specific human nuclear antigen antibody, with no negative cells observed (FIG. 5).

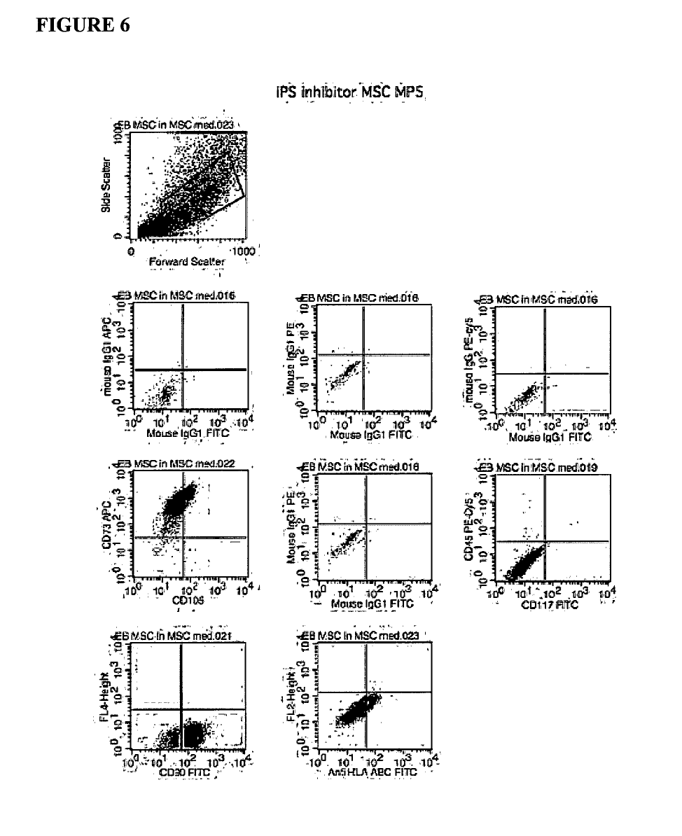

The SB431542 inhibitor differentiation method was also applied to the human iPSC line, ES4CL1, to produce MSC-like cells with characteristic fibroblast-like morphology and an immunophenotype similar to primary MSC (FIGS. 1D and 6).

Differentiation of iPSC in the presence of SB431542 resulted in the decreased expression of the pluripotency markers (Oct4, Nanog, Stella, SSEA3, Tra 1-60 and Tra 1-81)—see FIG. 8. Nuclear expression of SSEA4 was observed in iPSC colonies and iPS-MSC (SB431542) at mp2. Cells gained expression of a mesodermal marker, vimentin during differentiation from iPSC to iPS-MSC (inhibitor), indicating that iPSC had undergone differentiation into iPS-MSC. Nuclei were counter-stained with dapi (blue).

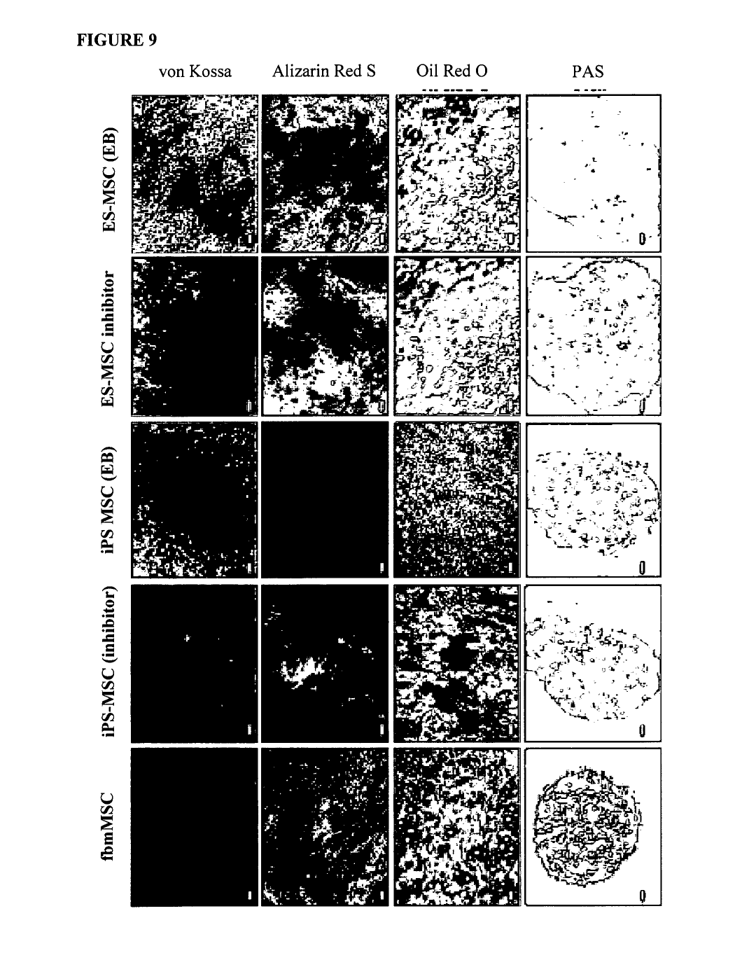

Mesodermal differentiation of fMSC, iPS-MSC and ES-MSC (inhibitor and EB) after 28 days stained with von Kossa, Alizarin Red S, Oil Red O and PAS (see FIG. 9).

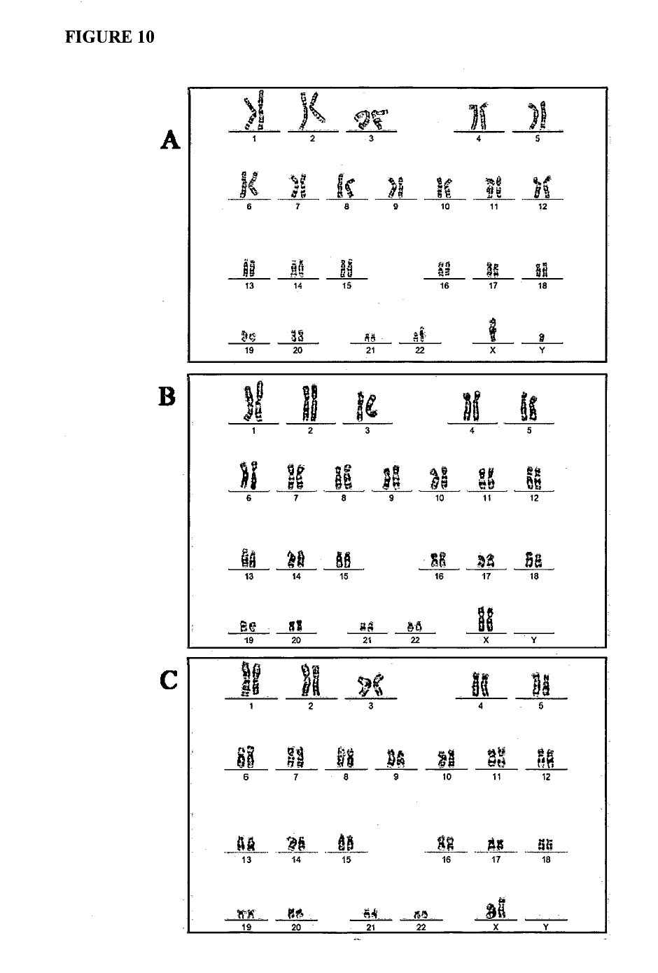

A karyotypic assessment of fMSC and iPS-MSC (generated by the inhibitor method and the EB method) demonstrated chromosome metaphase spreads showing normal karyotype of fMSC, iPS-MSC (inhibitor) and iPS-MSC (EB), analysed at early and late passages (see FIG. 10; mesenchymal passage 11-12 shown).

Further analysis showed that no teratomas formed in animals injected with iPS-MSC nine weeks after injection (see FIG. 11). In contrast, two teratomas formed in animals injected with iPSC, growing to 8 mm diameter seven weeks after injection. Histological analysis of haematoxylin and eosin stained teratoma sections revealed the presence of cells derived from the 3 germ layers: mesoderm (see FIGS. 11B and E), endoderm (FIGS. 11C and F) and ectoderm (FIGS. 11D and G).

Pluripotent hESC (MEL1) were also cultured in mTESR medium and KOSR medium with 10 μM SB431542 for 10 days. After 10 days treatment, MEL1 cells were subcultured in the MSC medium for differentiation into MSC. The MEL1-derived MSC were designated MEL1-MSC P2 (“P2” meaning passaged twice). After 10 days, SB431542-treated cells and MEL1-MSC P2 cells were analyzed for gene expression (see FIG. 12). OCT4, SOX2, MYST2 and EPCAM genes were associated with iPSC pluripotecy. NCAM, MSX2, LEFTY1 and BMP4 genes were expressed by cell lineages from the mesoderm. The PAX6 gene was expressed by ectoderm and the CDX2 gene was expressed by trophectoderm. GATA gene was expressed by cell lineages from the endoderm. Genes expressed by cell lineages from MSC were positive for CD29, CD73 and negative for CD117, CD133.

Pluripotent iPSC (MR90) were cultured in mTESR medium and KOSR medium with 10 μM SB431542 for 10 days. After 10 days, MR90 cells were subcultured in MSC medium (DMEM-HG with 10% foetal bovine serum) for differentiating MSC. The MEL1-derived MSC were designated MR90-MSC P0 (“P0” meaning passage 0). After 10 days, SB431542-treated cells and MR90-MSC P0 cells were analyzed for gene expression (see FIG. 13). OCT4, SOX2, MYST2 and EPCAM genes were associated with iPSC pluripotecy. NCAM, MSX2, LEFTY1 and BMP4 genes were expressed by cell lineages from the mesoderm. The PAX6 gene was expressed by ectoderm and the CDX2 gene was expressed by trophectoderm. GATA gene was expressed by cell lineages from the endoderm. Genes expressed by cell lineages from MSC were positive for CD29, CD73 and negative for CD117, CD133.

Analysis was performed by flow cytometry for EPCAM expression in the SB431542-treated MR90 cells after at 10 days in culture. The analysis revealed 45% EPCAM+ MR90 cells in the mTESR culture (see FIG. 14).

When ESC/iPSC cells were cultured in mTeSR and KOSR condition medium with 10 μM SB431542 for 10 days, then subcultured into MSC medium to differentiate cells into MSC, gene expression profiling revealed the expression of genes defining all three germ layers and MSC markers (see FIG. 15).

Throughout this specification and the claims which follow, unless the context requires otherwise, the word “comprise”, and variations such, as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

1. A method for generating mesenchymal stem cells from a population of embryonic stem cells (ESC) or induced pluripotent stem cells (iPS), the method comprising:
(i) differentiating a population of ESC or iPS attached to a surface of a culture vessel by exposing the cells to an inhibitor of endogenous activin and TGF-β signalling to produce a monolayer of cells comprising epithelial cell-like morphology attached to the surface of the culture vessel; and
(ii) passaging the cells differentiated in step (i) in the presence of a mesenchymal stem cell medium for a time and under conditions sufficient to produce mesenchymal stem cells.

2. The method of claim 1, wherein the inhibitor of endogenous activin and TGF-β signalling is 4-[4-(1,3-benzodioxol-5-yl)-5-(2-pyridinyl)-1H-imidazol-2-yl]-benzamide (SB431542).

3. The method of claim 1, wherein the ESC or iPS are differentiated in step (i) in the presence of an attachment factor.

4. The method of claim 1, wherein step (i) comprises differentiating a population of iPS.

5. A composition comprising the mesenchymal cell or population of mesenchymal cells generated by the method according to claim 1.

6. The composition according to claim 5 further comprising a pharmaceutically acceptable carrier.

7. The composition according to claim 5 further comprising a tissue matrix.

8. A method for treating a subject in need thereof comprising administering to the subject the composition of claim 5, claim 6 or claim 7.

9. (canceled)

10. A method for generating mesenchymal stem cells from a population of ESC or iPS, the method comprising:
(i) differentiating a population of ESC or iPS in the presence of an inhibitor of endogenous activin and TGF-β signalling under conditions sufficient to inhibit formation of embryoid bodies (EB); and
(ii) passaging the cells differentiated in step (i) in the presence of a mesenchymal stem cell medium for a time and under conditions sufficient to produce mesenchymal stem cells.

11. The method of claim 10, wherein the ESC or iPS are attached to a surface of a culture vessel and the cells differentiated in step (i) produce a monolayer of cells comprising epithelial cell-like morphology attached to the surface of the culture vessel.

12. The method of claim 11, wherein the ESC or iPS are differentiated in step (i) in the presence of an attachment factor.

13. The method of claim 10, wherein the inhibitor of endogenous activin and TGF-β signalling is 4-[4-(1,3-benzodioxol-5-yl)-5-(2-pyridinyl)-1H-imidazol-2-yl]-benzamide (SB431542).

14. The method of claim 10, wherein step (i) comprises differentiating a population of iPS.

15. A composition comprising the mesenchymal cell or population of mesenchymal cells generated by the method according to claim 10.

16. The composition according to claim 15 further comprising a pharmaceutically acceptable carrier.

17. The composition according to claim 15 further comprising a tissue matrix.

18. A method for treating a subject in need thereof comprising administering to the subject the composition according to claim 15, 16 or 17.

19. (canceled)

20. The method of claim 8, wherein the subject is a human.

21. The method of claim 8, wherein the subject is a veterinary subject.

22. The method of claim 18, wherein the subject is a human.

23. The method of claim 18, wherein the subject is a veterinary subject.
